(19) United States
(12) Patent Application Publication
(10) Pub. No.: US 2020/0381129 A1
(43) Pub. Date: Dec. 3, 2020

(54) SYSTEMS AND METHODS FOR PROVIDING CLINICAL TRIAL STATUS INFORMATION FOR PATIENTS

(71) Applicant: Flatiron Health, Inc., New York, NY (US)

(72) Inventors: Alexander Padmos, New York, NY (US); Angel Leung, Brooklyn, NY (US); Dominic Green, Brooklyn, NY (US); Eitan Meir Konigsburg, South Orange, NJ (US); Janet Donegan, Park City, UT (US); Lauren Sutton, New York, NY (US); Nathan Chan, New York, NY (US); Raman Choudhry, New York, NY (US); Victor J. Wang, New York, NY (US)

(73) Assignee: Flatiron Health, Inc.

(21) Appl. No.: 16/887,605

(22) Filed: May 29, 2020

Related U.S. Application Data
(60) Provisional application No. 62/853,933, filed on May 29, 2019.

(51) Int. Cl. G16H 50/70; G16H 10/60; A61B 5/00

(52) U.S. Cl. CPC G16H 50/70; A61B 5/7435; A61B 5/743; G16H 10/60

(57) ABSTRACT

A system for mining trial information from electronic medical records may include a processor programmed to analyze electronic medical records (EMRs) to determine first patients associated with a trial and a first trial status during a time period; analyze the EMRs to determine second patients associated with the trial and a second trial status during the time period; analyze the EMRs to determine third patients associated with a status change during the time period. The status change includes a change from a pre-screening status to the first trial status during the time period. The at least one processor may also be programmed to cause a display to display a graphical user interface configured to display a representation of a number of the first patients, a representation of a number of the second patients, and a representation of a number of the third patients.

### CROSS-REFERENCE TO RELATED APPLICATION

This application claims the benefit of priority of U.S. Provisional Patent Application No. 62/853,933, filed May 29, 2019, the disclosure of which is incorporated herein by reference in its entirety.

### BACKGROUND

### Technical Field

The present disclosure relates to systems and methods for mining and providing trial status information of patients.

### Background Information

Identifying patients who are eligible for clinical trials and track the statuses of patient trials are the challenges the medical research community faces. While there are reasons that may dissuade a patient from participating in clinical trials, there are also many barriers. For example, identifying a patient at just the right time such as, for instance, when they are ready to be put on a therapy but have not yet started one, is often challenging when a practice may have dozens of trials open, each with a dozen or more inclusion/exclusion criteria, and with hundreds of patients coming into a practice a day. Thus, to overcome these challenges faced by existing systems, it is desirable to identify eligible patients for a clinical trial and eligible trials for a patient more efficiently. Additionally, it is desirable to track the statuses of the patients for a trial to improve trial recruitment and management, which may benefit both patients and researchers. Moreover, it is desirable to provide research coordinators and/or health care service providers an improved graphical user interface for displaying trial status information for one or more trials. Further, it is desirable to provide systems and methods with improved workflows between research coordinators and health care service providers by automatically sharing updated trial information among them, which may significantly increase the number of patients participating in clinical trials and benefit both patients and the medical study community.

### SUMMARY

Embodiments consistent with the present disclosure include systems and methods for mining and providing trial information related to one or more patients. The trial information may include status information for the patients with respect to a particular trial.

In an embodiment, a system for mining trial information from electronic medical records may include at least one processor programmed to analyze a plurality of electronic medical records to determine a first plurality of patients associated with the medical trial and a first trial status during a time period. The at least one processor may also be programmed to analyze the plurality of electronic medical records to determine a second plurality of patients associated with the medical trial and a second trial status during the time period. The at least one processor may further be programmed to analyze the plurality of electronic medical records to determine a third plurality of patients associated with a status change during the time period. The status change includes a change from a pre-screening status to the first trial status during the time period. The at least one processor may also be programmed to cause a display to display a graphical user interface comprising at least a first area and a second area. The first area may be configured to display a representation of a number of the first plurality of patients associated with the first trial status and a representation of a number of the second plurality of patients associated with the second trial status. The second area may be configured to display a representation of a number of the third plurality of patients associated with the status change.

In an embodiment, a computer-implemented method for mining trial information from electronic medical records may include analyzing a plurality of electronic medical records to determine a first plurality of patients associated with the medical trial and a first trial status during a time period. The method may also include analyzing the plurality of electronic medical records to determine a second plurality of patients associated with the medical trial and a second trial status during the time period. The method may further include analyzing the plurality of electronic medical records to determine a third plurality of patients associated with a status change during the time period. The status change includes a change from a pre-screening status to the first trial status during the time period. The method may also include causing a display to display a graphical user interface comprising at least a first area and a second area. The first area may be configured to display a representation of a number of the first plurality of patients associated with the first trial status and a representation of a number of the second plurality of patients associated with the second trial status. The second area may be configured to display a representation of a number of the third plurality of patients associated with the status change.

In an embodiment, a non-transitory computer-readable medium may include instructions that, when executed by at least one processor, cause the at least one processor to analyze a plurality of electronic medical records to determine a first plurality of patients associated with the medical trial and a first trial status during a time period. The instructions may also cause the at least one processor to may also be programmed to analyze the plurality of electronic medical records to determine a second plurality of patients associated with the medical trial and a second trial status during the time period. The instructions may also cause the at least one processor to analyze the plurality of electronic medical records to determine a third plurality of patients associated with a status change during the time period. The status change includes a change from a pre-screening status to the first trial status during the time period. The instructions may further cause the at least one processor to cause a display to display a graphical user interface comprising at least a first area and a second area. The first area may be configured to display a representation of a number of the first plurality of patients associated with the first trial status and a representation of a number of the second plurality of patients associated with the second trial status. The second area may be configured to display a representation of a number of the third plurality of patients associated with the status change.

The foregoing general description and the following detailed description are exemplary and explanatory only and are not restrictive of the claims.

### BRIEF DESCRIPTION OF THE DRAWINGS

The accompanying drawings, which are incorporated in and constitute part of this specification, and together with the description, illustrate and serve to explain the principles of various exemplary embodiments. In the drawings:

FIG. 1A is a block diagram illustrating an exemplary system for mining and providing trial status information of patients, consistent with the present disclosure.

FIG. 1B is a block diagram illustrating an exemplary computing device for mining and providing trial status information of patients, consistent with the present disclosure.

FIG. 2 is a diagram illustrating an exemplary graphical user interface for displaying trial information, consistent with the present disclosure.

FIG. 3 is a diagram illustrating an exemplary graphical user interface for displaying trial information, consistent with the present disclosure.

FIG. 4 is a diagram illustrating an exemplary graphical user interface for displaying trial information, consistent with the present disclosure.

FIG. 5 is a diagram illustrating an exemplary data structure of electronic medical records, consistent with the present disclosure.

FIG. 6 is a flowchart illustrating an exemplary process for mining trial status information of patients, consistent with the present disclosure.

### DETAILED DESCRIPTION

The following detailed description refers to the accompanying drawings. Wherever possible, the same reference numbers are used in the drawings and the following description to refer to the same or similar parts. While several illustrative embodiments are described herein, modifications, adaptations and other implementations are possible. For example, substitutions, additions or modifications may be made to the components illustrated in the drawings, and the illustrative methods described herein may be modified by substituting, reordering, removing, or adding steps to the disclosed methods. Accordingly, the following detailed description is not limited to the disclosed embodiments and examples. Instead, the proper scope is defined by the appended claims.

Embodiments herein include computer-implemented methods, tangible non-transitory computer-readable mediums, and systems. The computer-implemented methods may be executed, for example, by at least one processor (e.g., a processing device) that receives instructions from a non-transitory computer-readable storage medium. Similarly, systems consistent with the present disclosure may include at least one processor (e.g., a processing device) and memory, and the memory may be a non-transitory computer-readable storage medium. As used herein, a non-transitory computer-readable storage medium refers to any type of physical memory on which information or data readable by at least one processor may be stored. Examples include random access memory (RAM), read-only memory (ROM), volatile memory, non-volatile memory, hard drives, CD ROMs, DVDs, flash drives, disks, and any other known physical storage medium. Singular terms, such as “memory” and “computer-readable storage medium,” may additionally refer to multiple structures, such a plurality of memories and/or computer-readable storage mediums. As referred to herein, a “memory” may comprise any type of computer-readable storage medium unless otherwise specified. A computer-readable storage medium may store instructions for execution by at least one processor, including instructions for causing the processor to perform steps or stages consistent with an embodiment herein. Additionally, one or more computer-readable storage mediums may be utilized in implementing a computer-implemented method. The term “computer-readable storage medium” should be understood to include tangible items and exclude carrier waves and transient signals.

In this disclosure, systems and method for providing trial status information relating to patients for a trial are provided. In some embodiments, a system may mine electronic medical records associated with patients to provide trial status information of a trial. The system may analyze the electronic medical records to determine a first plurality of patients associated with a medical trial. The first plurality of patients may have a first trial status for the trial (e.g., a screening status) during a period of time (e.g., in January). The system may also analyze the electronic medical records to determine a second plurality of patients who have a second trial status (e.g., an active status) for the same trial during the same period of time. The system may further display in a graphical user interface the information relating to the first plurality of patients and the second plurality of patients. In some embodiments, the system may also receive and analyze an updated electronic medical record associated with a patient to automatically update the trial status information.

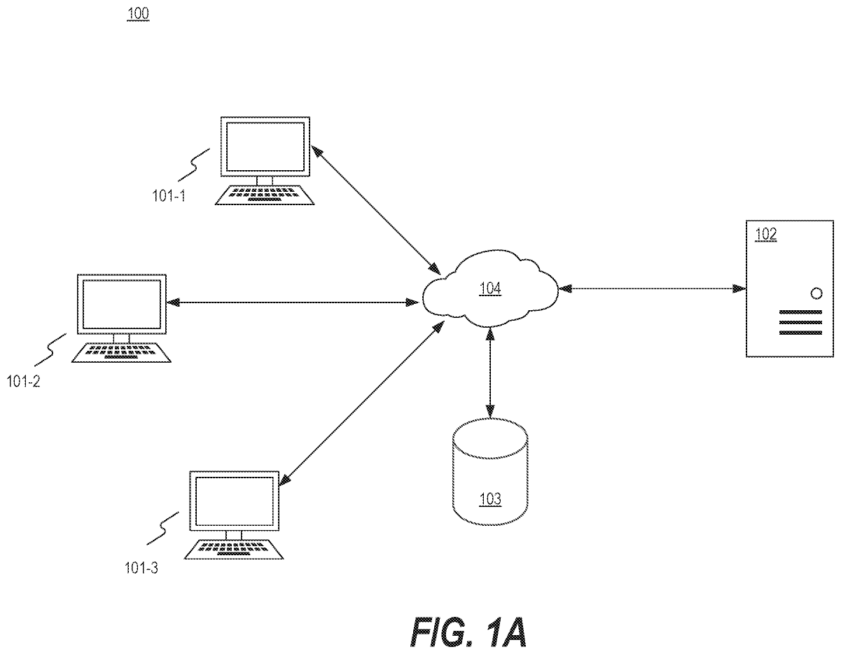

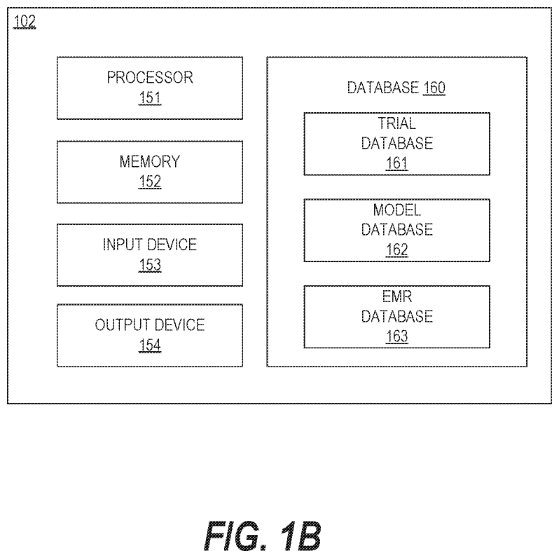

FIG. 1A illustrates an exemplary system 100 for implementing embodiments consistent with the present disclosure. As shown in FIG. 1A, system 100 may include one or more client devices 101, a computing device 102, a database 103, and a network 104. It will be appreciated from this disclosure that the number and arrangement of these components are exemplary and provided for purposes of illustration. Other arrangements and numbers of components may be used without departing from the teachings and embodiments of the present disclosure.

A client device 101 (e.g., client device 101-1, 101-2, 101-3) may be configured to collect and enter information relating to one or more patients. For example, client device 101 may reside at a clinic, and a user (e.g., a physician or administrator) may enter information relating to a patient visit at an input device of client device 101. As another example, a researcher may update a trial status of a patient for a trial (e.g., from a “CANDIDATE” status to an “SIGNED MAIN STUDY CONSENT” status). The information received by client device 101 may be transmitted to computing device 102, which may associate the information with one or more electronic medical records associated with the patient. In some embodiments, client device 101 may also receive and present information received from computing device 102. For example, client device 101 may receive information relating to trial information relating to one or more patients from computing device 102 and present the information at an interface of client device 101 to the user. In some embodiments, client devices 101-1, 101-2, and 101-2 may reside at the same site or different sites. In some embodiments, the information received by client device 101 may include clinic notes, radiology reports, pathology reports, doctor or nurse observations, structured and unstructured data, and any other type of information that may be included in a patient's medical record (e.g., an electronic medical record or other available data sources, claims data, patient-reported data). As used herein, “unstructured” refers to text that is not categorized into one or more standardized formats (e.g., a date format, a name format, or the like) and is not in a serialized or markup format (such as XML, YAML, JSON, or the like). In one embodiment, the unstructured data may be captured by an abstraction process, while the structured data may be entered by the health care professional or calculated using algorithms. In one embodiment, data sources 430 may include medical care providers (e.g., physicians, hospitals), laboratories, insurance companies, and any other source of patient data.

Computing device 102 may be configured to mine electronic medical records to determine and provide trial status information for a trial. For example, computing device 102 may analyze the electronic medical records to determine a first group of patients having a first trial status over last 30 days and a second group of patients having a second trial status over the same period. The first trial status may be different from the second trial status. In some embodiments, the first trial status (and the second trial status) may include one of a PRE-CONSENT status, a CONSENTED status, and an ENROLLED status. A PRE-CONSENT status may include one of a WATCHING status, a CANDIDATE status, an IN PRE-SCREENING status, a DEEMED INELIGIBLE status, and an IN CONSENTING PROCESS status. A CONSENTED Status may include one of a SIGNED MAIN STUDY CONSENT status and a SCREEN FAIL status. An ENROLLED status may include one of an ON TRIAL status (which may include one of an ACTIVE status and an IN FOLLOW UP status) and an OFF TRIAL status (which may include one of a COMPLETED status and a DISCONTINUATION status).

In some embodiments, computing device 102 may automatically generate an algorithm for suggesting one or more eligible patients for a trial based on trial eligibility criteria. By way of example, computing device 102 may create a namedtuple that has numbers and a series of letters for each of the patients based on the electronic medical record (e.g., age, disease, biomarkers). Computing device 102 may evaluate the created namedtuples associated with the patients against the expression tree, which may return a number indicating the eligibility for each of the patients. For example, the expression-tree algorithm may output “0” for ineligible or “1” for eligible. Alternatively, the algorithm may output a probability value indicating the eligibility for each of the patients. A screening trial status may be assigned to the suggested patients.

In some embodiments, client device 101 and computing device 102 may be integrated into one device configured to perform the functions of client device 101 and computing device 102 disclosed in this application. For example, a user may enter information relating to a patient for a trial via input device 153 of computing device 102. Computing device 102 may update the trial status information (if needed) based on the received information and display the updated trial status information.

Database 103 may be configured to store information and data for one or more components of system 100. For example, database 103 may store electronic medical records associated with one or more patients. Database 103 may also store information relating to one or more trials. In some embodiments, database 103 may also store patient-trial matching algorithms for determining one or more suggested eligible patients for a trial, and/or one or more suggested eligible trials for a patient. Client device 101 and/or computing device 102 may be configured to access and obtain the data stored on database 103 via network 104. In some embodiments, database 103 may be operated by a third party. For example, computing device 102 may request information relating to a particular trial from database 103, which may transmit the requested information to computing device 102.

Network 104 may be configured to facilitate communications among the components of system 100. Network 104 may include a local area network (LAN), a wide area network (WAN), portions of the Internet, an Intranet, a cellular network, a short-ranged network (e.g., a Bluetooth™ based network), or the like, or a combination thereof.

FIG. 1B is a block diagram illustrating an exemplary computing device 102. Computing device 102 may include at least one processor (e.g., processor 151), a memory 152, an input device 153, an output device 154, and a database 160.

Processor 151 may be configured to perform one or more functions described in this application. Computing device 102 may also include a memory 152 that may store instructions for various components of computing device 102. For example, memory 152 may store instructions that, when executed by processor 151, may be configured to cause processor 151 to perform one or more functions described herein.

Input device 153 may be configured to receive input from the user of computing device 102, and one or more components of computing device 102 may perform one or more functions in response to the input received. In some embodiments, input device 153 may include an interface displayed on a touchscreen (e.g., output device 154). Output device 154 may be configured to output information and/or data to the user. For example, output device 154 may include a display configured to display the information relating to a trial. In some embodiments, output device 154 may include a touchscreen. As another example, computing device 102 may generate a report including information relating to a trial based on the electronic medical records. Output device 154 may output the generated report.

Database 160 may be configured to store various data and information for one or more components of computing device 102. For example, database 160 may include a trial database 161, a model database 162, and an electronic medical record (EMR) database 163. Trial database 161 may be configured to store information relating to one or more trials. For example, trial database 161 may store a trial portfolio for each of the trials, which may include trial eligibility criteria of a trial. Trial eligibility criteria of a trial may include a trial status, a trial disease, a trial line of therapy, an eligibility age, a trial biomarker criterion, or the like, or a combination thereof. In some embodiments, a trial portfolio may also include trial name, trial description, or the like, or a combination thereof. Trial database 161 may further store edit history including changes made to a trial. Computing device 102 may obtain information relating to the trials from trial database 161 and modify the information if needed. For example, computing device 102 may create a trial portfolio for a new trial and store the trial portfolio into trial database 161.

Model database 162 may store patient-trial matching models or algorithms. A patient-trial matching algorithm refers to an algorithm for determining one or more eligible patients for a trial and/or for determining one or more suggested eligible trials for a patient. Computing device 102 may obtain algorithms from model database 162. In some embodiments, computing device 102 may create an algorithm for a new trial and store the created algorithm into model database 162. EMR database 163 may store electronic medical records associated with patients. Processor 151 may receive one or more electronic medical records from EMR database 163.

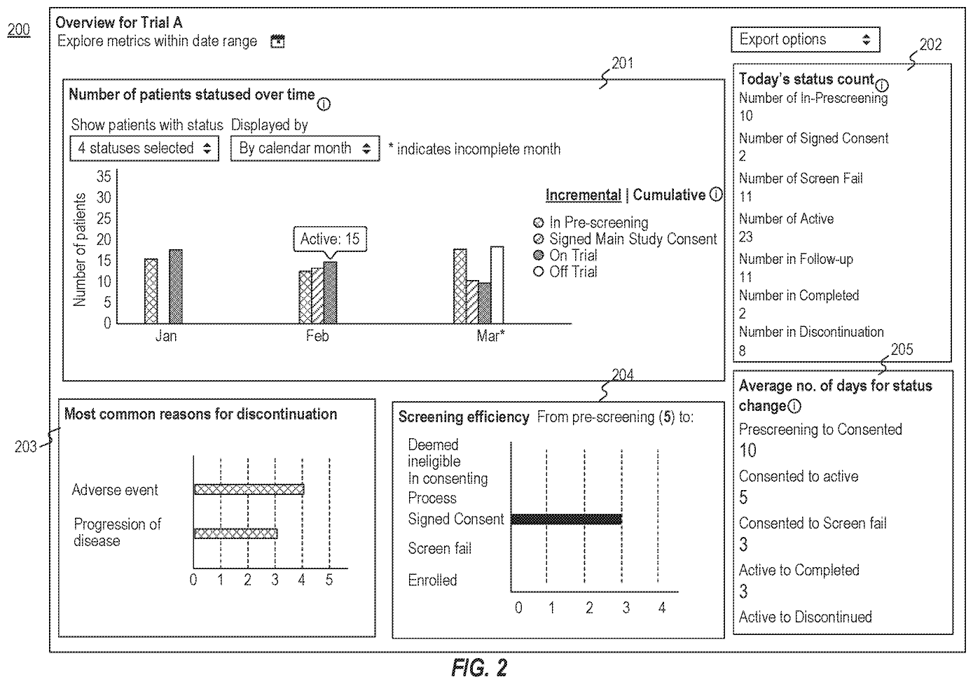

FIG. 2 is a diagram illustrating an exemplary graphical user interface 200 for viewing trials, consistent with the present disclosure. User interface 200 may be displayed via output device 154 of computing device 102 (e.g., a display or touchscreen). Alternatively or additionally, computing device 102 may transmit instructions to client device 101 for displaying graphical user interface 200 via an output device of client device 101 (e.g., a display or touchscreen). Computing device 102 may obtain information from a database (e.g., database 103, database 160) and render graphical user interface 200 based on the obtained information.

User interface 200 may include a first area 201 configured to display trial information relating to a plurality of patients. For example, the first area 201 may include a bar chart including representations of the numbers of patients having various statuses over different time periods. Computing device 102 may analyze a plurality of electronic medical records to determine a first plurality of patients who have a first trial status (e.g., the IN-PRE-SCREEN status) in a first time period (e.g., January) and determine a second plurality of patients who have a second trial status (e.g., the ON TRIAL status) in the first time period. Computing device 102 may also be configured to cause a display to present user interface 200 including representations of the number of the first plurality of patients and the number of the second plurality of patients in January. For example, user interface 200 may include a bar chart to illustrate the number of the patients having an IN-PRE-SCREEN status (i.e., the first trial status in this example) and the number of the patients having an ON TRIAL status (i.e., the second trial status in the example) in January.

In some embodiments, the user may select one or more statuses for which information may be displayed in user interface 200. For example, the user may be configured to select one or more trial status from a dropdown, which may include a plurality of statuses described in this application. After the user selects one or more statuses, user interface 200 may display the number of the statuses that the user selected (e.g., by displaying the text “4 statuses selected” illustrated in FIG. 2). In some embodiments, selecting a parent status may trigger selecting a default child status. Alternatively or additionally, if a parent status is unselected, all its child statuses may be unselected. In some embodiments, a predetermined set of statuses may be selected by default. For example, a set of statuses including IN PRE-SCREENING, SIGNED MAIN STUDY CONSENT, SCREEN FAIL, ENROLLED may be selected by default.

In some embodiments, the user may set a date range for displaying the trial information relating to the data range. For example, user interface 200 may include two date selectors, one for a “Start Date” and one for an “End Date”. In some embodiments, computing device 102 may select a default date range for start date as a predetermined number of months (e.g., three or four months) before today (e.g., January to March illustrated in user interface 200), and default for end date is today. In some embodiments, modifying the date selector by the user may cause all data in the user interface to filter by statuses or data created within the selected date range.

In some embodiments, computing device 102 may determine the time period over which the number of the first plurality of patients and the number of the second plurality of patients are displayed in user interface 200 based on the data range. For example, computing device 102 may determine January (or the first 30 days of the date range) as the first time period and determine February (or the second 30 days of the date range) as the second time period. Alternatively, computing device 102 may determine the first time period and the second time period (and one or more time periods) based on a date grouping selected by the user through user input. Exemplary date groupings may include a time period of 7 days, 14 days, 30 days, or calendar month (e.g., January, February), or the like. In some embodiments, the calendar month may be selected by default. Computing device 102 may also cause user interface 200 to display the numbers of the patients having the first trial status in January and February, and the numbers of the patients having the second trial status in January and February (as represented in various bars in the bar chart illustrated in FIG. 2). In some embodiments, the bars may have different colors, each of which may correspond to a particular status.

In some embodiments, for each selected status or status group, user interface 200 display a bar on the bar chart, according to the time periods grouped by the time range (e.g., January, February, March) indicated by the date grouping. In some embodiments, if a parent of statuses is selected, such as “PRE-CONSENT,” user interface 200 may show a bar for the parent group. The count may be the sum of counts in the children statuses. The same may apply for the “ENROLLED” grandparent group. For example, when the “ENROLLED” grandparent group is selected, user interface 200 may show “Enrolled” as one bar, and children bars will not be shown. In some embodiments, the enrollment goal for the trial may be displayed as a line so that the user may see whether the goal is reached with ease.

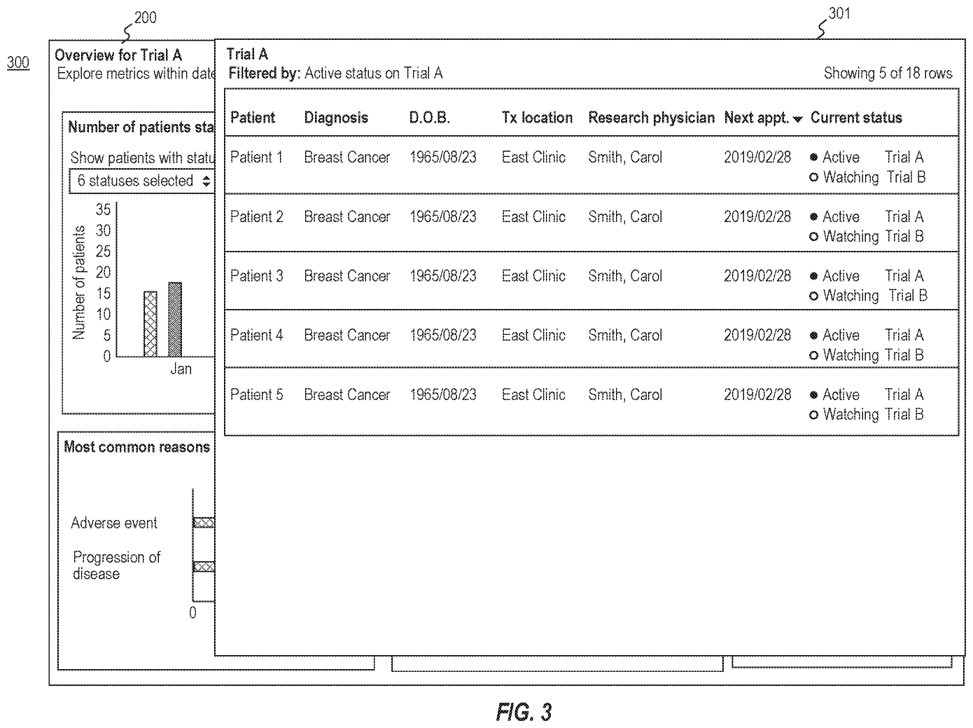

In some embodiments, user interface 200 may be configured to interact with the user. For example, the user may move a cursor hover over a bar representing the number of the patients having a particular state, user interface 200 may automatically display the number corresponding to that status (i.e., the text “Active 15” illustrated in FIG. 2). As another example, the user may click a bar, and user interface 200 user interface 200 may open an overlay, which may partially overlap with first area 201 of user interface 200, showing a list of patients who have the corresponding status (such as a patient list 301 shown in graphical user interface 300 illustrated in FIG. 3) during the corresponding time period.

In some embodiments, if a patient has multiple statuses in the same time period, only the most recent status will be counted. e.g., if patient has “WATCHING” on January 15, and “CANDIDATE” on January 30, and the date grouping was “calendar month,” the patient will be counted only as “CANDIDATE.”

In some embodiments, user interface 200 may include a second area 202 configured to display the numbers of patients having various current statuses. Second area 202 may represent a snapshot of the current patient states. Each patient may only contribute to one count. For example, a patient who had an ACTIVE state last week, but is in follow-up this week may contribute to the FOLLOW-UP status count, and may not contribute to the ACTIVE state count. In some embodiments, second area 202 mays not affected by the date range selector. Second area 202 may display one or more of the current number of patients having a IN-PRESCREENING status, the current number of patients having a SCREENING status, the current number of patients having an ACTIVE status, the current number of patients having a FOLLOW-UP status, the current number of patients having a COMPLETED status, and the current number of patients having a DISCONTINUED status. In some embodiments, computing device 102 may be configured to receive user input to interact with the numbers displayed (or a symbol or text associated therewith). For example, a user may click a number, user interface 200 may open an overlay, showing a list of patients who have the corresponding status (e.g., the patient list illustrated in illustrated in FIG. 3).

In some embodiments, user interface 200 may include a third area 203 configured to show information relating to one or more statuses. For example, third area 203 may show the most common reasons why the patients have a particular status (e.g., a DISCONTINUATION status illustrated in FIG. 2). In some embodiments, computing device 102 may analyze the electronic medical records to determine the most common reasons for a status. For example, computing device 102 may analyze the relevant notes from physicians (and/or researchers) and determine the reasons why the trial is discontinued for one or more patients. Computing device 102 may also rank the reasons to determine the most common reasons (e.g., based on the numbers of the patients for each of the reasons). In some embodiments, computing device 102 may consider discontinuing statuses with a date within the selected date range as described elsewhere in this disclosure. In some embodiments, user interface 200 may display the reasons and their counts on a horizontal bar graph. Alternatively or additionally, user interface 200 may be configured to interact with the user's input for generating a report for a reason selected by the user.

In some embodiments, user interface 200 may include a fourth area 204 configured to show a screening efficiency. The screening efficiency may be represented by the numbers of the patients who had a PRE-SCREEN status and changed to various statuses during a time period. The numbers and graph may capture, for those patients that had a pre-screening status, what status they move onto. For example, as illustrated in FIG. 2, fourth area 204 may display the numbers of patients with various statuses between the date range. In some embodiments, computing device 102 may only consider patients that have one of these statuses within the selected date range. In some embodiments, if a patient has a CANDIDATE status added before the date range, but that status did not change within the date range, this patient should be counted as having a CANDIDATE status. In some embodiments, computing device 102 may only consider the status that immediately follows a screening status (e.g., WATCHING, CANDIDATE, IN PRE-SCREENING). In some embodiments, user interface 200 may be configured to interact with the user based on the user's input. For example, if the user moves a cursor on a bar, user interface 200 may display the count for that bar. As another example, if a particular number is clicked on, user interface 200 may open an overlay showing a list of patients under that metric (e.g., patient list 301 illustrated in illustrated in FIG. 3).

In some embodiments, user interface 200 may include an area 205 configured to show the average number of days for status change for various statuses. For example, computing device 102 may determine the patients that have both status A and status B (e.g., status B has a date that is equal to or a later date than status A), and determine the average number of days for status change from status A to status B. Computing device 102 may also be configured to cause user interface 200 to display the calculated number. By way of example, user interface 200 may include the average number of days for the status changes from at least one of IN PRE-SCREENING to CONSENT, CONSENT to ACTIVE, CONSENT to SCREEN FAIL, ACTIVE to COMPLETED, and ACTIVE to DISCONTINUED. In some embodiments, a displayed number may be the average number of days between status A's date and status B's date, rounded up to the nearest whole number. In some embodiments, the number of days should not include the day of status A, but include the day of status B.

FIG. 3 is a diagram illustrating an exemplary graphical user interface 300 for displaying trial information, consistent with the present disclosure. User interface 300 may include an overlap over user interface 200 showing a patient list of the patients having one or more particular statuses over a time period. For example, as described elsewhere in this disclosure, the user may interact with user interface 200 by clicking a number (or a bar) relating to a particular status, user interface 300 may display an overlay on user interface 200 showing a patient list of the patients having the particular status over the time period.

In some embodiments, in the overlay, a table is shown with the list of patients, which may include various columns, such as information relating to a patient (e.g., the patient's name, medical record number), diagnosis, treating location, research physician, next appointment, current status on trial(s) (status+trial name), or the like, or a combination thereof. In some embodiments, a title text may be displayed for what the metric is, which may be specified for each metric that can be clicked on. Alternatively or additionally, an “Export” button may be displayed for generating a spreadsheet file (e.g., a csv or Excel file) of the list of patients for the user.

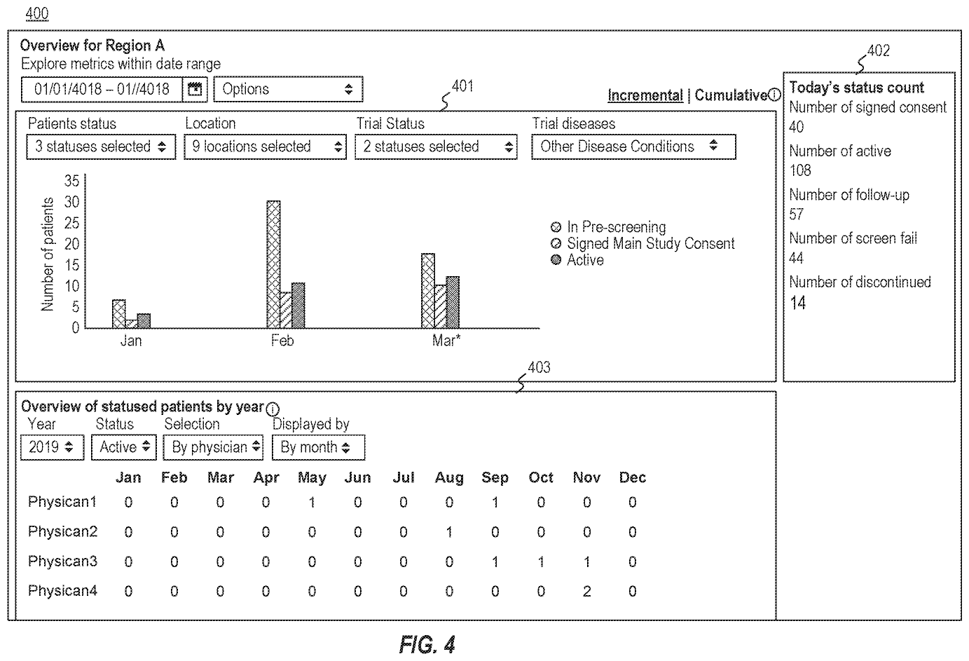

FIG. 4 is a diagram illustrating an exemplary graphical user interface 400 for displaying trial information relating to a region (or a treatment location), consistent with the present disclosure. User interface 400 may include a first area 401 to show trial information similar to user interface 200 (illustrated in FIG. 2), but for the patients in a particular region or a treatment location. For example, as illustrated in FIG. 4, first area 401 may include representation of the numbers of the patients at Region A who have an IN PRE-SCREENING status in January, February, and March. As another example, user interface 400 may include a second area 402 showing the current status counts for various statuses in Region A.

In some embodiments, user interface 400 (and/or user interface 200) may also include an area 403 showing the numbers of trial-patients where a patient has an ACTIVE status (or a FOLLOW UP, DISCONTINUATION, or COMPLETED status) with a status date within the selected time range. As illustrated in FIG. 4, the numbers may be grouped according to particular research physicians, which may be determined by computing device 102 based on an attribute included in relevant electronic medical records (e.g., a trial_patient_attribute). In some embodiments, the numbers may be displayed as a horizontal bar graph, ordered by frequency.

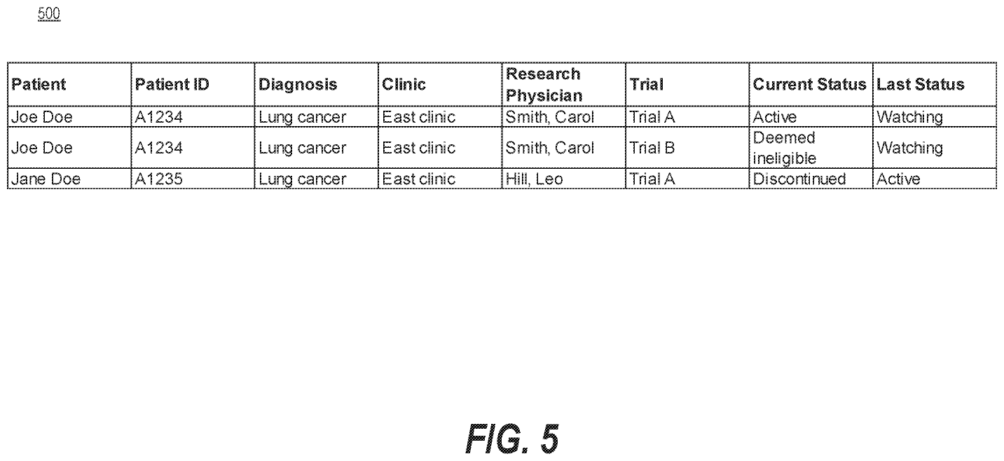

FIG. 5 is a diagram illustrating an exemplary data structure 500 of electronic medical records, consistent with the present disclosure. In some embodiments, computing device 102 may store information relating to a patient and a trial as an entry according to data structure 500. Data structure 500 may include a table having various fields such as Patient, Patient ID, Diagnosis, Clinic, Research Physician, Trial, Current Status, Last Status, etc. Each of the fields may store information including numeric or alphanumeric characters. In some embodiments, computing device 102 may analyze the electronic medical records to determine the first (and/or second) plurality of patients by accessing the table storing patient information for the trial, which may include at least a first plurality of fields storing patient identifiers (e.g., patient's name and/or ID), a second plurality of fields storing an identifier of the first (and/or second) trial status (e.g., ACTIVE), and a third plurality of fields storing an identifier of the time period (e.g., January).

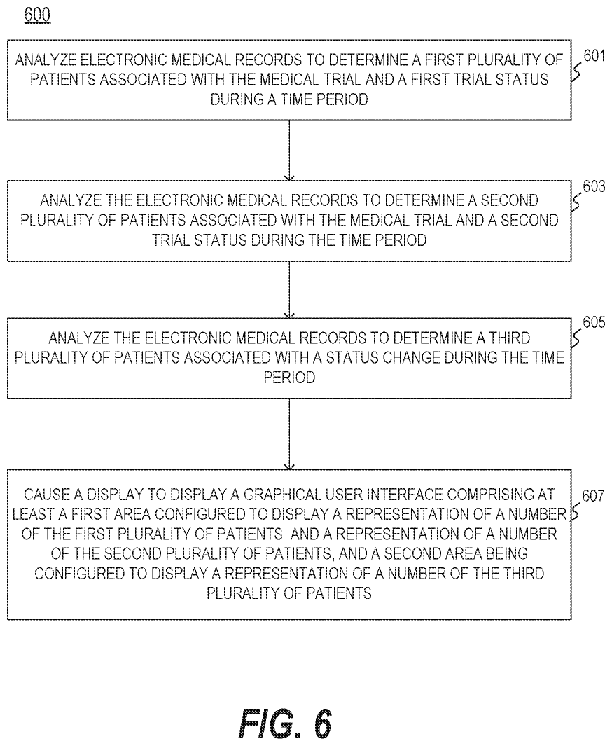

FIG. 6 is a flowchart illustrating an exemplary process 600 for mining trial status information of patients, consistent with the present disclosure. At step 601, computing device 102 may analyze a plurality of electronic medical records to determine a first plurality of patients associated with the medical trial and a first trial status during a time period. For example, computing device 102 may access and obtain the electronic medical records stored in a database (e.g., database 103). Computing device 102 may also determine the patients who have a first trial status (e.g., a status of being ineligible for the trial, a status of the trial being discontinued for a patient, or a status that a patient has failed a screening, or a status described elsewhere in this disclosure) over the time period.

In some embodiments, computing device 102 may access a table storing patient information for the trial, which may include a data structure similar to data structure 500 illustrated in FIG. 5. For example, computing device 102 may store information relating to a patient and a trial as an entry according to data structure 500. Data structure 500 may include a table having various fields such as Patient, Patient ID, Diagnosis, Clinic, Research Physician, Trial, Current Status, Last Status, etc. In some embodiments, computing device 102 may analyze the electronic medical records to determine the first (and/or second) plurality of patients by accessing the table storing patient information for the trial, which may include at least a first plurality of fields storing patient identifiers (e.g., patient's name and/or ID), a second plurality of fields storing an identifier of the first (and/or second) trial status (e.g., ACTIVE), and a third plurality of fields storing an identifier of the time period (e.g., January). In some embodiments, the table may include at least a first plurality of fields storing patient identifiers and a second plurality of fields storing an identifier of a trial status associated with a corresponding patient. Computing device 102 may determine, based on the first plurality of fields and the second plurality of fields, the third plurality of patients having the status change from the pre-screening status to the first trial status during the time period.

At step 603, computing device 102 may be configured to analyze the plurality of electronic medical records to determine a second plurality of patients associated with the medical trial and a second trial status during the time period. Computing device 102 may be configured to analyze the electronic medical records to determine the second plurality of patients who have a second trial status for the trial during the time period similar to step 601. For example, computing device 102 may analyze the electronic medical records stored in a data structure similar to data structure 500 to determine the second) plurality of patients by accessing the table storing patient information for the trial.

At step 605, computing device 102 may be configured to analyze the plurality of electronic medical records to determine a third plurality of patients associated with a status change during the time period. The status change may include a change from a PRE-SCREENING status to the first trial status during the time period. For example, computing device 102 may determine a third plurality of patients who had a PRE-SCREENING status and changed the status to another status during the time period based on the electronic medical records. By way of example, computing device 102 may access a table storing patient information for the trial, which may include at least a first plurality of fields storing patient identifiers and a second plurality of fields storing an identifier of a trial status associated with a corresponding patient. Computing device 102 may also determine, based on the first plurality of fields and the second plurality of fields, the third plurality of patients having the status change from the pre-screening status to the first trial status during the time period.

At step 607, computing device 102 may be configured to cause a display to display a graphical user interface comprising at least a first area and a second area. The first area may be configured to display a representation of a number of the first plurality of patients associated with the first trial status and a representation of a number of the second plurality of patients associated with the second trial status. The second area may be configured to display a representation of a number of the third plurality of patients associated with the status change. For example, computing device 102 may be configured to cause the display to second area 201 of user interface 200 illustrated in FIG. 2 (as described elsewhere in this disclosure), showing a representation of the number of the first plurality of patients and a representation of the number of the second plurality of patients in January. In some embodiments, the representation of the number of the first plurality of patients (and/or the second plurality of patients) may include numerical text, alphanumerical text, a symbol, one or more words, a graphical representation (e.g., a bar, an image, a shape, etc.), or the like, or a combination thereof.

In some embodiments, the graphical user interface may include a bar graph comprising a first bar representing the number of the first plurality of patients associated with the first trial status, and a second bar representing of the number of the second plurality of patients associated with the second trial status. In one embodiment, the first bar may be in a first color and the second bar is in a second color different from the first color.

In some embodiments, the graphical user interface may include a pie chart comprising a first slice and a second slice. The first slice may represent the number of the first plurality of patients associated with the first trial status, and the second slice may represent the number of the second plurality of patients associated with the second trial status.

In some embodiments, the graphical user interface further comprises a third area configured to display a representation of a current number of patients having the first trial status (such as second area 202 of user interface 200 illustrated in FIG. 2).

In some embodiments, as illustrated in FIG. 2, computing device 102 may be configured to analyze the plurality of electronic medical records to determine a fourth plurality of patients associated with the medical trial and the first trial status (e.g., an IN PRE-SCREENING status) during a second time period (e.g., in February), which may be different from the first time period (e.g., in January). Computing device 102 may also be configured to analyze the plurality of electronic medical records to determine a fifth plurality of patients associated with the medical trial and the second trial status (e.g., an ON TRIAL status) during the second time period. Computing device 102 may further be configured to analyze the plurality of electronic medical records to determine a sixth plurality of patients associated with the status change from the PRE-SCREENING status to the first trial status (e.g., an IN PRE-SCREENING status) during the second time period. In some embodiments, the user interface may include a first area showing a representation of the number of the fourth plurality of patients and a representation of a number of the fifth plurality of patients. The user interface may also include a second area showing a representation of the number of the sixth plurality of patients.

In some embodiments, the graphical user interface may include information relating to one or more reasons why one or more patients have the first trial status. For example, as illustrated in FIG. 2, user interface 200 may include a third area 203 configured to the most common reasons why the patients have a particular status (e.g., a DISCONTINUATION status).

In some embodiments, computing device 102 may generate a report including trial status information. For example, computing device 102 may be configured to generate a report comprising information relating to one or more reasons why at least one of the first plurality of patients has the first trial status. In some embodiments, the user interface may include an “Export” button or dropdown menu for outputting a report including trial status information described elsewhere in this disclosure. For example, an “Export” dropdown menu may include options for generating a report including at least one of a Patient List, a Screening Log, and Reasons relating to why one or more patients have a particular status (e.g., DEEMED INELIGIBLE, SCREEN FAIL, DISCONTINUATION, etc.). For example, the user may select export “Reasons for Deemed Ineligible,” and computing device 102 may generate a report with all the patients having a DEEMED INELIGIBLE status. The report may include a spreadsheet such as an Excel file, which may include various columns such as patient name+MRN in parenthesis, discontinuation date, discontinuation reason, or the like, or a combination thereof.

The foregoing description has been presented for purposes of illustration. It is not exhaustive and is not limited to the precise forms or embodiments disclosed. Modifications and adaptations will be apparent to those skilled in the art from consideration of the specification and practice of the disclosed embodiments. Additionally, although aspects of the disclosed embodiments are described as being stored in memory, one skilled in the art will appreciate that these aspects can also be stored on other types of computer readable media, such as secondary storage devices, for example, hard disks or CD ROM, or other forms of RAM or ROM, USB media, DVD, Blu-ray, 4K Ultra HD Blu-ray, or other optical drive media.

Computer programs based on the written description and disclosed methods are within the skill of an experienced developer. The various programs or program modules can be created using any of the techniques known to one skilled in the art or can be designed in connection with existing software. For example, program sections or program modules can be designed in or by means of .Net Framework, .Net Compact Framework (and related languages, such as Visual Basic, C, etc.), Java, Python, R, C++, Objective-C, HTML, HTML/AJAX combinations, XML, or HTML with included Java applets.

Moreover, while illustrative embodiments have been described herein, the scope of any and all embodiments having equivalent elements, modifications, omissions, combinations (e.g., of aspects across various embodiments), adaptations and/or alterations as would be appreciated by those skilled in the art based on the present disclosure. The limitations in the claims are to be interpreted broadly based on the language employed in the claims and not limited to examples described in the present specification or during the prosecution of the application. The examples are to be construed as non-exclusive. Furthermore, the steps of the disclosed methods may be modified in any manner, including by reordering steps and/or inserting or deleting steps. It is intended, therefore, that the specification and examples be considered as illustrative only, with a true scope and spirit being indicated by the following claims and their full scope of equivalents.

What is claimed is:

1. A system for mining trial information from electronic medical records, comprising:
at least one processor programmed to:
analyze a plurality of electronic medical records to determine a first plurality of patients associated with the medical trial and a first trial status during a time period;
analyze the plurality of electronic medical records to determine a second plurality of patients associated with the medical trial and a second trial status during the time period;
analyze the plurality of electronic medical records to determine a third plurality of patients associated with a status change during the time period, wherein the status change includes a change from a pre-screening status to the first trial status during the time period; and
cause a display to display a graphical user interface comprising at least a first area and a second area, the first area being configured to display a representation of a number of the first plurality of patients associated with the first trial status and a representation of a number of the second plurality of patients associated with the second trial status, and the second area being configured to display a representation of a number of the third plurality of patients associated with the status change.

2. The system of claim 1, wherein analyzing the plurality of electronic medical records to determine the first plurality of patients includes accessing a table storing patient information for the trial, wherein the table includes at least a first plurality of fields storing patient identifiers, a second plurality of fields storing an identifier of the first trial status, and a third plurality of fields storing an identifier of the time period.

3. The system of claim 1, wherein analyzing the plurality of electronic medical records to determine the second plurality of patients includes accessing a table storing patient information for the trial, wherein the table includes at least a first plurality of fields storing patient identifiers, a second plurality of fields storing an identifier of the first trial status, and a third plurality of fields storing an identifier of the time period.

4. The system of claim 1, wherein analyzing the plurality of electronic medical records to determine the third plurality of patients associated with the status change comprises:
accessing a table storing patient information for the trial, wherein the table includes at least a first plurality of fields storing patient identifiers and a second plurality of fields storing an identifier of a trial status associated with a corresponding patient; and
determining, based on the first plurality of fields and the second plurality of fields, the third plurality of patients having the status change from the pre-screening status to the first trial status during the time period.

5. The system of claim 1, wherein the plurality of electronic medical records are stored in a database.

6. The system of claim 1, wherein the first trial status includes a status of being ineligible for the trial.

7. The system of claim 1, wherein the first trial status includes a status of the trial being discontinued for a patient.

8. The system of claim 1, wherein the first trial status includes a status that a patient has failed a screening.

9. The system of claim 1, the graphical user interface comprises information relating to one or more reasons why one or more patients have the first trial status.

10. The system of claim 1, wherein the representation of the number of the first plurality of patients includes numerical text.

11. The system of claim 1, wherein the representation of the number of the second plurality of patients includes a graphical representation.

12. The system of claim 1, wherein the graphical user interface comprises a bar graph comprising a first bar and a second bar, and the first bar representing the number of the first plurality of patients associated with the first trial status, the second bar representing of the number of the second plurality of patients associated with the second trial status.

13. The system of claim 12, wherein the first bar is in a first color and the second bar is in a second color.

14. The system of claim 1, wherein:
the first trial status includes at least one of a status of being ineligible for the trial, a status of the trial being discontinued for a patient, or a status that a patient has failed a screening; and
the at least one processor is further programmed to generate a report comprising information relating to one or more reasons why at least one of the first plurality of patients has the first trial status.

15. The system of claim 1, wherein the at least one processor is further programmed to:
analyze the plurality of electronic medical records to determine a fourth plurality of patients associated with the medical trial and the first trial status during a second time period;
analyze the plurality of electronic medical records to determine a fifth plurality of patients associated with the medical trial and the second trial status during the second time period; and
analyze the plurality of electronic medical records to determine a sixth plurality of patients associated with the status change from the pre-screening status to the first trial status during the second time period, wherein:
the first area comprises a representation of a number of the fourth plurality of patients and a representation of a number of the fifth plurality of patients, and
the second area comprises a representation of a number of the sixth plurality of patients.

16. The system of claim 1, wherein the graphical user interface further comprises a third area configured to display a representation of a current number of patients having the first trial status.

17. The system of claim 1, wherein the time period is determined based on an input by a user.

18. The system of claim 1, wherein at least one processor is further programmed to:
receive a user input relating to the first plurality of patients; and
cause, based on the user input, the graphical user interface to display a third area comprising patient information of at least one of the first plurality of patients, wherein the third area at least partially overlaps with the first area or the second area.

19. A computer-implemented method for mining trial information from electronic medical records, comprising:
analyzing a plurality of electronic medical records to determine a first plurality of patients associated with the medical trial and a first trial status during a time period;
analyzing the plurality of electronic medical records to determine a second plurality of patients associated with the medical trial and a second trial status during the time period;
analyzing the plurality of electronic medical records to determine a third plurality of patients associated with a status change during the time period, wherein the status change includes a change from a pre-screening status to the first trial status during the time period; and
causing a display to display a graphical user interface comprising at least a first area and a second area, the first area being configured to display a representation of a number of the first plurality of patients associated with the first trial status and a representation of a number of the second plurality of patients associated with the second trial status, and the second area being configured to display a representation of a number of the third plurality of patients associated with the status change.

20. A non-transitory computer-readable medium comprising instructions that, when executed by at least one processor, cause the at least one processor to:
analyze a plurality of electronic medical records to determine a first plurality of patients associated with a medical trial and a first trial status during a time period;
analyze the plurality of electronic medical records to determine a second plurality of patients associated with the medical trial and a second trial status during the time period;
analyze the plurality of electronic medical records to determine a third plurality of patients associated with a status change during the time period, wherein the status change includes a change from a pre-screening status to the first trial status during the time period; and
cause a display to display a graphical user interface comprising at least a first area and a second area, the first area being configured to display a representation of a number of the first plurality of patients associated with the first trial status and a representation of a number of the second plurality of patients associated with the second trial status, and the second area being configured to display a representation of a number of the third plurality of patients associated with the status change.
